(19) United States
(12) Patent Application Publication
(10) Pub. No.: US 2019/0066822 A1
(43) Pub. Date: Feb. 28, 2019

(54) SYSTEM AND METHOD FOR CLINICAL TRIAL MANAGEMENT

(71) Applicant: Elements of Genius, Inc., Mt. Pleasant, SC (US)

(72) Inventor: Jonathan E. Ramaci, Mt. Pleasant, SC (US)

(21) Appl. No.: 16/119,924

(22) Filed: Aug. 31, 2018

Related U.S. Application Data
(60) Provisional application No. 62/552,771, filed on Aug. 31, 2017.

(51) Int. Cl. G16H 10/20; G16H 10/60

(52) U.S. Cl. CPC G16H 10/20; G16H 10/60

(57) ABSTRACT

The present invention relates to computing devices, microcontrollers, memory storage devices, executable codes, methods, application software, automated voice recognition-response device, natural language understanding-processing methods, algorithms, risk stratification tools, and communication channels for conducting clinical trials. Embodiments of the present disclosure may function in combination with an application software accessible to multiple clients (users) executable on a remote server to provide participant education, support, enhance compliance to protocol, social contact, management of daily activities, safety monitoring, symptoms management, adverse events reporting, electronic data capture, as well as support for caregivers, and feedback for investigators and administrators in the management of clinical trials. Alternative embodiments implementing monitoring and intervention include using mobile apps or voice-controlled speech interface devices to access cloud control services capable of processing automated voice recognition-response and natural language understanding-processing to perform functions and fulfill user requests.

### CROSS REFERENCE TO RELATED APPLICATIONS

This application claims the benefit of U.S. Provisional Application 62/552,771, entitled “COLLABORATIVE ECOSYSTEM FOR THE MANAGEMENT OF CLINICAL TRIALS,” filed Aug. 31, 2017 and hereby incorporated by reference.

### FIELD

The present disclosure relates to the field of wearable connected systems for medical and pharmaceutical applications; in particular, a system and method for clinical trial management via a networked interface of wearable devices.

### BACKGROUND

A clinical trial (e.g., Randomized Controlled Trial (RCT) is a type of scientific (often medical/clinical) study which aims to reduce bias when testing a new treatment. The RCT is generally considered the gold standard for a clinical trial. RCTs are often used to test the safety (e.g., drug reactions) and efficacy (i.e., effectiveness) of various types of medical intervention, in the commercialization of prescription products (e.g., new chemical entities (NCEs), biologics, etc.). The development effort of drugs to treat chronic and degenerative diseases requires longer clinical trials to observe relevant outcomes. Clinical trial protocols have become increasingly complex, involving numerous assessments, exploratory endpoints, biomarkers, and the like, consequently increasing the administrative burden and overall costs of trials. Another significant trend contributing to higher clinical trial costs is the increased use of health care cost containment strategies, cost-effectiveness data requirements, and collecting patient-reported outcomes (PROs). These factors and regulatory barriers contribute to clinical trials with large numbers of patients and extended durations, resulting in greater expenditures on recruitment efforts, data collection, compliance with administrative requirements, and other trial components.

mHealth or eHealth is defined as medical and public health practices supported by mobile devices (e.g., mobile phones, patient monitoring devices, personal digital assistants, other wireless devices, and smartphones). Smartphone-assisted RCTs are an emerging methodology enabling new opportunities for the delivery of health interventions for research purposes and clinical evaluations of drugs. The advantages of smartphone-assisted health include the ability to deliver an intervention remotely and expanding the capability to reach a potentially large population. The use of smartphones ranges from sending simple information or text message reminders to participants to more complex tools enabling, for example, self-monitoring, and data collection; increasing being implemented via apps. In contrast to classical clinical visits, apps can be used for remote trial documentation of safety and potentially efficacy of a therapeutic intervention (e.g., NCE, biologic). Greater participant retention, protocol compliance, adherence to the intervention, and convenience for participants compared to traditional methods have been reported for smartphone-assisted RTCs.

Patient-reported outcomes (PROs) are increasingly emphasized in clinical trials (e.g., RTCs) and population health studies. They are self-report instruments that directly measure the patient's perceptions of the impact of disease and treatment, without interpretation of the patient's response by a clinician or anyone else, as clinical trial end points, especially for subjective symptoms. The use of PROs is particularly common for therapeutic interventions being developed to treat chronic, disabling conditions where the goal is not necessarily to cure but to ameliorate symptoms, facilitate functioning, or improve quality of life (QoL). PROs are the primary endpoints in clinical trials evaluating drug products for disease areas such as irritable bowel syndrome, migraine, and pain. In addition, PROs provide key supportive data in many other disease areas, such as insomnia, asthma, and psychiatric disorders. In oncology, PROs are commonly used to assess both treatment benefits and toxicity to fully evaluate the impact of treatment on health-related quality of life (HRQoL). PROs can also be used in clinical trials to assess treatment satisfaction, compliance, and caregiver burden. In general, scientifically validated PRO questionnaires may need to be completed repeatedly for weeks or months. However, there are several limitations of PROs and among these are their un-blinded nature and potential expectance bias. Questionnaires for assessing QoL are prone to being influenced by more than just disability. Other factors that are commonly seen in patients contribute as well (e.g. fatigue, depression, anxiety, and physical comorbidities). In addition, PROs are prone to response shift over time occurring when a patient answers an item differently from their previous responses, due to a change of internal standards, values or conceptualization of the purposed domain (e.g., QoL).

PROs require collecting data directly from patients themselves and are traditionally collected through face-to-face interviews and written questionnaires. The inherent weaknesses with these methods include high costs and burdensome data management. Computer-assisted tools have been developed for the collection of PROs. However, these tools are often only used in hospitals or clinics, making it a challenge to collect PROs when the patients are at home. Electronic diaries (eDiaries) have been adapted for use in RCTs. However, various issues have been attributed to the use of bulky eDiaries and the use of non-validated methods.

Electronic Data Capture (EDC) has a distinct advantage over paper-based systems of research, able to detect protocol violations and data outside the normal range at the time of entry. EDC systems have been shown to improve the quality of clinical trials, halt the development of ineffective or unsafe drugs earlier, reduce unnecessary work, reduce cost, and accelerate time to market of new drugs. There are also benefits in terms of data quality, performance, productivity and costs in clinical trial management. It is well accepted by users and has been shown to contribute to patient empowerment, allowing them to be more engaged in research and to take direct control of their own data. By contrast, the use of paper-based questionnaires can result with incomplete forms, considered time consuming, require dual checking, and data cleansing. Whereas, EDC can alert people to missing answers, and is easily incorporated into electronic health records. Remote data collection offers convenience to patients and may provide a safer environment for questionnaires than paper-based methods eliciting the answers to potentially sensitive questions. Mobile surveys may serve as a better form of Electronic Data Capture (EDC), enabling respondents to send pictures, record their voice, or write notes/diaries all on smartphones. Smartphone apps may be more efficient tools for longitudinal studies, where patients need to respond to questions repeatedly during RTCs. However, smartphones are currently not implemented in the convenient form of a wearable device. The management and execution of RCTs, even with the emergence of smart-phones, places unique challenges for participants (i.e., subjects), caregivers (e.g., family members), clinical investigators (e.g. physicians), and administrators. These challenges include the complexities and costs of clinical trial protocols, subject adherence, self-monitoring, data collection, measurement of clinical outcomes (e.g., primary & secondary, PROs outcomes to support product claims), and participants-investigator(s) interactions-communication. Despite the advantages, EDC has not been universally accepted and the perceived disadvantages and concerns include: complexity of installation, maintenance of software, high initial investment cost, complexity of use, and a lack of investigator motivation. The impact of tools for participant-physician communication have shown improvement on such outcomes as patient self-efficacy, satisfaction with care, and on clinical outcomes. However, few tools exist with the goal of facilitating secure team-based communication, enabling the sharing of information across health events and settings, and promotion of collaboration. Therefore, the need exists for a comprehensive and integrated solution to conduct clinical trials (e.g., RCTs), preferably one that incorporates optimal tools (e.g., convenient voice-controlled relational agent for subject engagement and compliance, wearable EDC and communication devices, collaborative environment, etc.) within a clinical trial ecosystem. Such a system should provide a more convenient, efficient, and cost-effective methodology for conducting clinical trials.

Through applied effort, ingenuity, and innovation, Applicant has identified a number of deficiencies and problems with effective management of clinical trials. Applicant has developed a solution that is embodied by the present invention, which is described in detail below.

### SUMMARY

The following presents a simplified summary of some embodiments of the invention in order to provide a basic understanding of the invention. This summary is not an extensive overview of the invention. It is not intended to identify key/critical elements of the invention or to delineate the scope of the invention. Its sole purpose is to present some embodiments of the invention in a simplified form as a prelude to the more detailed description that is presented later.

In the broadest terms, the invention is a pervasive integrated assistive technology platform (system) incorporating one or more computing devices, microcontrollers, memory storage devices, executable codes, methods, software, automated voice recognition-response device, automated voice recognition methods, natural language understanding-processing methods, algorithms, risk stratification tools, and communication channels for management and execution of clinical trials. The system incorporates comprehensive and optimal methods for subject engagement, monitoring, data collection, and compliance with study objectives, protocols/procedures, as well facilitating communication with the clinical investigation team (e.g., physician, nurse, etc.). The platform incorporates a wearable device providing one or more features of medication adherence, voice, data, SMS reminders, alerts, location via SMS, and 911 emergency. The device may function in combination with an application software platform accessible to multiple clients (users) executable on one or more remote servers to provide a collaborative clinical study ecosystem. The device may function in combination with one or more remote servers, cloud control services capable of providing automated voice recognition-response, natural language understand-processing, applications for predictive algorithm processing, sending reminders, alerts, sending general and specific information relating to the clinical study. One or more components of the mentioned system may be implemented through an external system that incorporates a stand-alone speech interface device in communication with a remote server, providing cloud-based control service, to perform natural language or speech-based interaction with the user. The stand-alone speech interface device listens and interacts with a user to determine a user intent based on natural language understanding of the user's speech. The speech interface device is configured to capture user utterances and provide them to the control service. The control service performs speech recognition-response and natural language understanding-processing on the utterances to determine intents expressed by the utterances. In response to an identified intent, the controlled service causes a corresponding action to be performed. An action may be performed at the controlled service or by instructing the speech interface device to perform a function. The combination of the speech interface device and one or more applications executed by the control service serves as a relational agent. The relational agent provides conversational interactions, utilizing automated voice recognition-response, natural language processing, predictive algorithms, and the like, to perform functions, interact with the user (e.g., subject, family member, etc.), fulfill user requests, educate and inform user, monitor user compliance, collect data such as endpoints (e.g., primary, secondary), safety (e.g. adverse events), outcomes (e.g., PROs), and the like. In a preferred embodiment, the wearable device's form-factor is a hypoallergenic wrist watch, a wearable mobile phone, incorporating functional features that include, but are not limited to, medication reminder, voice, data, SMS text messaging, fall detection, step counts, location-based services, and direct 911 emergency access. In an alternative embodiment, the wearable device's form factor is an ergonomic and attachable-removable to-and-from an appendage or garment of a user as a pendant or the like. The wearable device may contain one or more microprocessor, microcontroller, micro GSM/GPRS chipset, micro SIM module, read-only memory device, memory storage device, I-O devices, buttons, display, user interface, rechargeable battery, microphone, CODEC, speaker, wireless transceiver, antenna, accelerometer, vibrating motor(output), preferably in combination, to function fully as a wearable mobile cellular phone. The said device enables communication with one or more remote servers capable of providing automated voice recognition-response, natural language understand-processing, predictive algorithm processing, reminders, alerts, general and specific information for the management of chronic pain. One or more components of the mentioned system may be implemented through an external system that incorporates a stand-alone speech interface device in communication with a remote server, providing cloud-based control service, to perform natural language or speech-based interaction with the user. The said device enables the participant (i.e., subject enrolled in a study) to access and interact with the said relational agent for compliance with a study objective, protocol, and procedures that include, but are not limited to, instructions, following dosing regimens, receiving reminders (e.g., medication), scheduling visits, reporting symptoms/adverse events, accessing educational information, accessing social support, and communicating with the clinical investigation team (e.g., principal investigator, nurse, etc.).

In another preferred embodiment, the wearable device can communicate with a secured HIPAA-compliant remote server. The remote server is accessible through one or more computing devices, including, but not limited to, desk-top, laptop, tablet, mobile phone, smart appliances (i.e., smart TVs), and the like. The remote server contains a clinical study support application software that include a database for storing participant and user (s) information, demographics, electronic master file, electronic case report forms, or the like. The application software provides a collaborative working environment for management and execution of the goals and objectives of a clinical study. The software environment allows for, but is not limited to, daily tracking of application usage, daily tracking of subject location, monitoring adherence to study protocol, storing and tracking health data (e.g., blood pressure, glucose, cholesterol, etc.), informed consent, storing subject daily diaries, storing biomarkers, displaying symptom trends and severity, sending-receiving text messages, sending-receiving voice messages, sending-receiving images/videos, streaming instructional videos, scheduling clinic visits, participant education information, caregiver education information, feedback to clinical investigation team, and the like. The application software can be used to store skills relating to the study objectives, protocols, and procedures. The application software may contain functions for predicting patient behaviors, non-compliance to protocol/therapy, functions for predicting symptom trends, or functions for predicting clinical outcomes. The application software may interact with an electronic health, medical record system, EDC software, or other clinical study software applications.

In an alternative embodiment, the said secured remote server is accessible using said stand-alone speech interface device or the speech interface is incorporated into one or more smart appliances, or mobile apps, capable of communicating with the same or another remote server, providing cloud-based control service, to perform natural language or speech-based interaction with the user, acting as said relational agent. The relational agent provides conversational interactions, utilizing automated voice recognition-response, natural language learning-processing, perform various functions and the like, to: interact with the user, fulfill user requests, educate, monitor study protocol/procedure compliance, provide one or more skills, ask one or more questions, collect clinical/outcomes data, storing responses/answers, perform predictive algorithms with user responses, determine health status and well-being, and provide suggestions for corrective actions including instructions for reporting adverse events, symptoms, protocol deviations, and the like.

In yet another embodiment, skills are developed and accessible through the relational agent. These skills include disease specific educational topics, nutrition, study protocol instructions, study procedures, instructions for taking medication, skills to improve medication adherence, skills to increase persistence, skills for recording, reporting or managing symptoms, proprietary developed skills, skills developed by another party, participant coping skills (e.g., with depression, anxiety, etc.), behavioral skills (e.g., CBT), skills for daily activities, skills for caring for participants, skills for caregivers to support participants, and other skills disclosed in the detailed embodiments of this invention.

In yet another embodiment, the user interacts with the relational agent via providing responses or answers to clinically validated questionnaires, instruments, or PROs. The questionnaires enable the monitoring of patient behaviors, medication compliance, medication adherence, medication persistence, wellness, symptoms (e.g., pain, etc.), adverse events monitoring, record/capture study endpoints, record/capture clinical outcomes, and the like. The responses or answers provided to the relational agent serve as input to one or more predictive algorithms to calculate a risk stratification profile and trends. Such a profile can provide an assessment for the need of any intervention required by either the participant, clinical investigation team members, caregivers, or family members. The relational agent facilitates real-time EDC for the clinical study.

An object of the present disclosure is an integrated clinical trial management system comprising a participant interface device operably engaged with a communications network, the participant interface device being configured to communicate a clinical trial protocol to a participant user, receive a voice input from the participant user in response to the clinical trial protocol, process a voice transmission from the voice input, and communicate the voice transmission over the communications network via at least one communications protocol; a remote server being operably engaged with the participant interface device via the communications network to receive the voice transmission, the remote server executing a control service comprising an automated speech recognition function, a natural-language processing function, and one or more application protocols, the one or more application protocols comprising communicating a clinical trial protocol to the participant interface device, communicating instructions associated with the clinical trial protocol to the participant interface device, and storing one or more participant-reported outcomes associated with the clinical trial protocol; and, a clinical trial administrator interface device being operably engaged with the remote server via the communications network, the clinical trial administrator interface device being operable to configure the plurality of clinical trial protocols and display the one or more participant-reported outcomes.

Another object of the present disclosure is an integrated clinical trial management system comprising a participant interface device operably engaged with a communications network, the participant interface device being configured to communicate a clinical trial protocol to a participant user, receive a voice input from the participant user in response to the clinical trial protocol, process a voice transmission from the voice input, and communicate the voice transmission over the communications network via at least one communications protocol; a remote server being operably engaged with the participant interface device via the communications network to receive the voice transmission, the remote server executing a control service comprising an automated speech recognition function, a user management function, a natural-language processing function, and one or more application protocols, the one or more application protocols comprising communicating a clinical trial protocol to the participant interface device, communicating instructions associated with the clinical trial protocol to the participant interface device, and storing one or more participant-reported outcomes associated with the clinical trial protocol; a clinical trial administrator interface device being operably engaged with the remote server via the communications network, the clinical trial administrator interface device being operable to configure the plurality of clinical trial protocols and display the one or more participant-reported outcomes; and, an investigator interface device being operably engaged with the remote server via the communications network, the investigator interface device being operable to configure one or more participant safety protocols.

Yet another object of the present disclosure is a method of clinical trial management comprising configuring, with a clinical trial administrator interface device, a plurality of clinical trial protocols associated with a clinical trial; configuring, with a remote server executing an application software, a plurality of application protocols corresponding to the plurality of clinical trial protocols; communicating, with the remote server via a communications network, one or more participant instructions corresponding to the plurality of clinical trial protocols to a participant interface device; gathering, with the participant interface device, one or more patient-reported outcomes corresponding to the plurality of clinical trial protocols, the one or more patient-reported outcomes comprising at least one voice input; communicating, with the participant interface device via a communications network, a voice transmission corresponding to the at least one voice input to the remote server; processing, with the application software executing on the remote server, the voice transmission to define a plurality of participant data, the plurality of participant data being stored in a database according to the plurality of application protocols; assessing, with the application software executing on the remote server, the plurality of participant data to define a plurality of participant safety and compliance data; and communicating, with the remote server via the communications network, one or more voice interaction prompts corresponding to the plurality of clinical trial protocols to the participant interface device.

In summary, the pervasive integrated assistive technology platform enables a high level of collaborative interaction for participants, clinical investigation team members, caregivers, and family members in the management and execution of clinical trial goals and objectives. The system leverages a voice-controlled empathetic relational agent for data capture/collection and to assist with subject engagement/participation in combination with a collaborative clinical study ecosystem to achieve superior study outcomes.

The foregoing has outlined rather broadly the more pertinent and important features of the present invention so that the detailed description of the invention that follows may be better understood and so that the present contribution to the art can be more fully appreciated. Additional features of the invention will be described hereinafter which form the subject of the claims of the invention. It should be appreciated by those skilled in the art that the conception and the disclosed specific methods and structures may be readily utilized as a basis for modifying or designing other structures for carrying out the same purposes of the present invention. It should be realized by those skilled in the art that such equivalent structures do not depart from the spirit and scope of the invention as set forth in the appended claims.

### BRIEF DESCRIPTION OF DRAWINGS

The above and other objects, features and advantages of the present disclosure will be more apparent from the following detailed description taken in conjunction with the accompanying drawings, in which:

FIG. 1 is a system diagram of a clinical trial management system incorporating a portable mobile device;

FIG. 2 is a system diagram of the clinical trial management system incorporating a wearable mobile device;

FIG. 3 is a perspective view of a wearable device and key features;

FIG. 4 depicts an alternate wearing option and charging function;

FIG. 5 is a graphical user interface containing the features of an application software platform providing a cognitive wellness ecosystem for implementing the clinical trial management system;

FIG. 6 depicts a graphical user interface of the application software platform accessed through a multimedia player-television using a voice-activated speech interface remote controlled device;

FIG. 7 illustrates the pervasive integrated assistive technology system incorporating a multimedia device;

FIG. 8 is a function block diagram of the elements of a relational agent; and,

FIG. 9 is a process flow diagram of a method for clinical trial management, according to an embodiment of the present disclosure.

### DETAILED DESCRIPTION

Exemplary embodiments are described herein to provide a detailed description of the present disclosure. Variations of these embodiments will be apparent to those of skill in the art. Moreover, certain terminology is used in the following description for convenience only and is not limiting. For example, the words “right,” “left,” “top,” “bottom,” “upper,” “lower,” “inner” and “outer” designate directions in the drawings to which reference is made. The word “a” is defined to mean “at least one.” The terminology includes the words above specifically mentioned, derivatives thereof, and words of similar import.

Embodiments of the present invention will now be described more fully hereinafter with reference to the accompanying drawings, in which some, but not all, embodiments of the invention are shown. Indeed, the invention may be embodied in many different forms and should not be construed as limited to the embodiments set forth herein; rather, these embodiments are provided so that this disclosure will satisfy applicable legal requirements. Where possible, any terms expressed in the singular form herein are meant to also include the plural form and vice versa, unless explicitly stated otherwise. Also, as used herein, the term “a” and/or “an” shall mean “one or more,” even though the phrase “one or more” is also used herein. Furthermore, when it is said herein that something is “based on” something else, it may be based on one or more other things as well. In other words, unless expressly indicated otherwise, as used herein “based on” means “based at least in part on” or “based at least partially on.” Like numbers refer to like elements throughout.

This disclosure describes a pervasive integrated assistive technology platform for facilitating a high level of interaction between study participants, clinical investigation team, caregivers, and family members in the management and execution of clinical studies. The system leverages a voice-controlled empathetic relational agent for patient education, patient support, patient social contact support, support of daily activities, patient safety, symptoms management, and support for caregivers, feedback/communication for the clinical investigation team. The platform enables the optimization of electronic data capture (EDC) leading optimal study outcomes. In one embodiment, the platform or system comprises a combination of at least one of the following components: communication device; computing device; communication network; remote server; cloud server; cloud application software. The cloud server and service are commonly referred to as “on-demand computing”, “software as a service (SaaS)”, “platform computing”, “network-accessible platform”, “cloud services”, “data centers,” and the like. The cloud server is preferably a secured HIPAA-compliant remote server. In an alternative embodiment, the intervention system comprises a combination of at least one; voice-controlled speech interface device; computing device; communication network; remote server; cloud server; cloud application software. These components are configured to function together to enable a user to interact with a resulting relational agent. In addition, an application software, accessible by the user and others, using one or more remote computing devices, provides a clinical study ecosystem, to enable an active and collaborative effort between study participants, clinical investigation team, caregivers, and family members, in a mutually acceptable manner to optimize and achieve superior clinical study results.

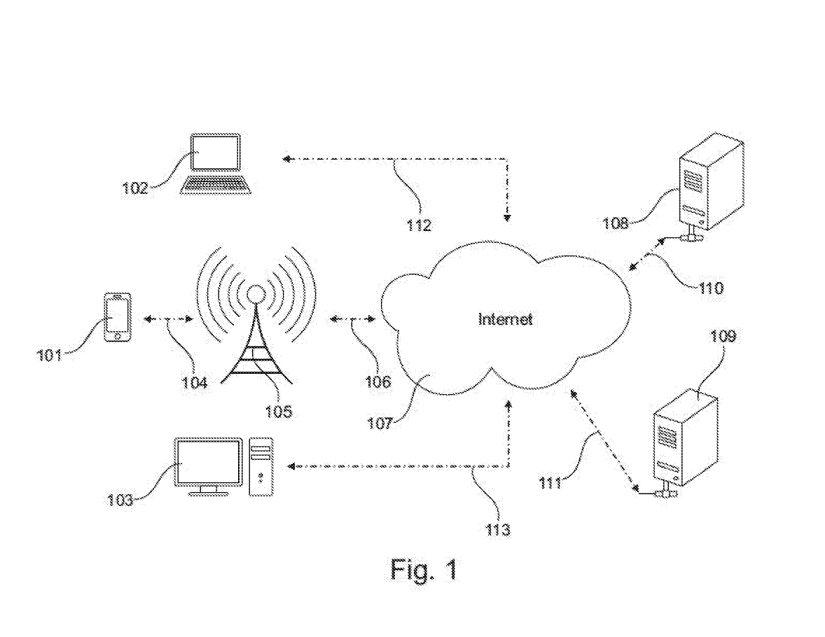

FIG. 1 illustrates the pervasive integrated assistive technology system incorporating a portable mobile device 101 for a study participant to interact with one or more remote clinical investigation team member, caregiver, or family member. One or more user can access the system using a portable computing device 102 or stationary computing device 103. Device 101 communicates with the system via communication means 104 to one or more cellular communication network 105 which can connect device 101 via communication means 106 to the Internet 107. Device 101, 102, and 103 can access one or more remote servers 108, 109 via the Internet 107 through communication means 110 and 111 depending on the server. Device 102 and 103 can access one or more servers through communication means 112 and 113. Computing devices 101, 102, and 103 are preferable examples but may be however any communication device, including tablet devices, cellular telephones, personal digital assistant (PDA), a mobile Internet accessing device, or other user system including, but not limited to, pagers, televisions, gaming devices, laptop computers, desktop computers, cameras, video recorders, audio/video player, radio, GPS devices, any combination of the aforementioned, or the like. Communication means may comprise hardware, software, communication protocols, Internet protocols, methods, executable codes, instructions, known to one of ordinary skill in the art, and combined to as to establish a communication channel between two or more devices. Communication means are available from one or more manufacturers. Exemplary communication means include wired technologies (e.g., wires, universal serial bus (USB), fiber optic cable, etc.), wireless technologies (e.g., radio frequencies (RF), cellular, mobile telephone networks, satellite, Bluetooth, etc.), or other connection technologies. The network useful to implement this invention is representative of any type of communication network, including data and/or voice networks, and may be implemented using wired infrastructure (e.g., coaxial cable, fiber optic cable, etc.), a wireless infrastructure (e.g. RF, cellular, microwave, satellite, Bluetooth, etc.), and/or other connection technologies.

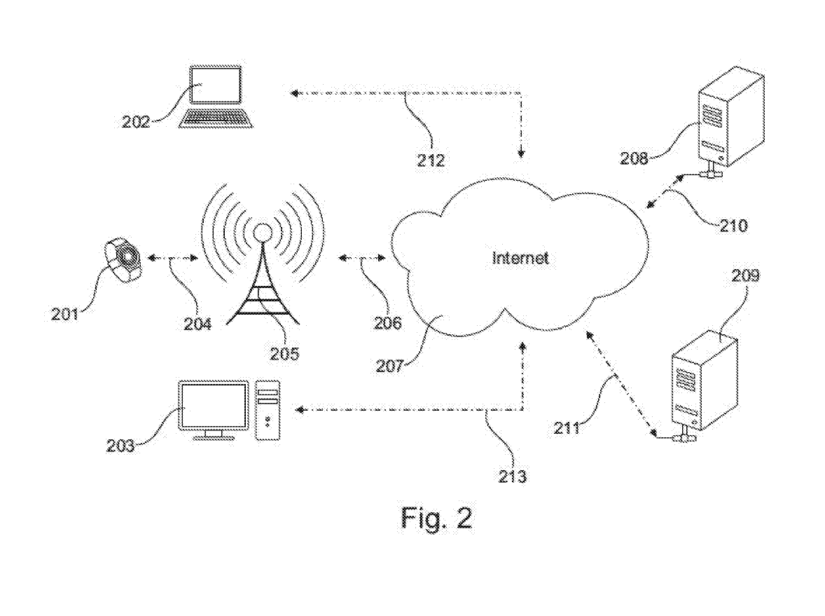

FIG. 2 illustrates the pervasive integrated assistive system incorporating a wearable device 201 for a study participant to interact with one or more remote clinical investigation team member, caregiver, or family member. In a similar manner as illustrated in FIG. 1, one or more user can access the system using a portable computing device 202 or stationary computing device 203. Computing device 202 may be a laptop used by a family member or caregiver. Stationary computing device 203 may reside at the facility of a clinical investigation team (e.g., principal investigator, nurse, CRO). Device 201 communicates with the system via communication means 204 to one or more cellular communication network 205 which can connect device 201 via communication means 206 to the Internet 207. Device 201, 202, and 203 can access one or more remote servers 208, 209 via the Internet 207 through communication means 210 and 211 depending on the server. Device 202 and 203 can access one or more servers through communication means 212 and 213.

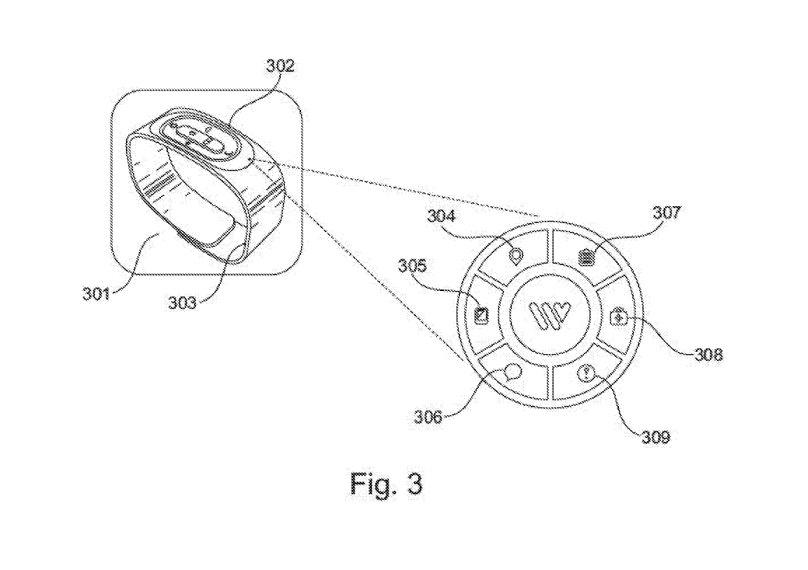

FIG. 3 is a pictorial rendering of the form-factor of a wearable device 301 as a wrist watch as a component of the pervasive integrated assistive technology system. The wearable device 301 is a fully functional mobile communication device (i.e., mobile cellular phone) that can be worn on the wrist of a user (e.g., participant). The wearable device 301 comprises a watch-like device 302 snap-fitted onto a hypoallergenic wrist band 303. The watch-like device 302 provides a user-interface that allows a user to access features that include smart and secure location-based services 304, mobile phone module 305, voice and data 306, advanced battery system and power management 307, direct 911 access 308, and fall detection accelerometer sensor 309. The wearable device may contain one or more microprocessor, microcontroller, micro GSM/GPRS chipset, micro SIM module, read-only memory device, memory storage device, I-O devices, buttons, display, user interface, rechargeable battery, microphone, CODEC, speaker, wireless transceiver, antenna, accelerometer, vibrating motor, preferably in combination, to function fully as a wearable mobile cellular phone. A study participant may use wearable device 301, depicted as device 201 of FIG. 2, to communicate with one or more clinical investigation team member, caregiver, or family member. The wearable device 301 may allow a participant to access one or more remote cloud servers to communicate with a relational agent. The relational agent facilitates real-time EDC for the clinical study.

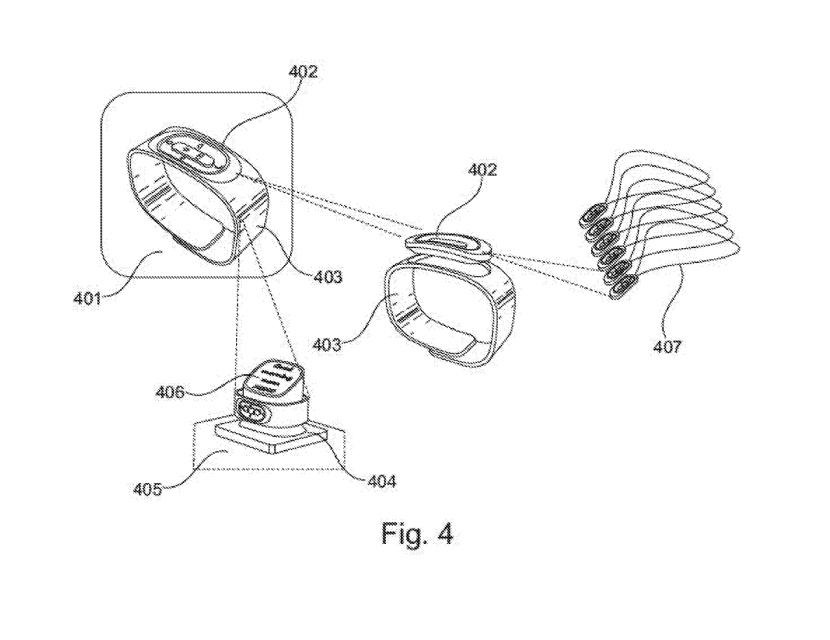

FIG. 4 illustrates details on additional features of the preferred wearable device. Wearable device 401 comprises a watch-like device 402 and wrist band 403, depicted in FIG. 3 as wearable device 301. Wearable device 401 can be stored together with a base station 404 and placed on top of platform 405. Platform 405 may be the surface of any furniture including a night stand. Base station 404 contains electronic hardware, computing devices, and software to perform various function, for example to enable the inductive charging of the rechargeable battery of wearable device 401, among others. Base station 404 also has a user interface 406 that can display visual information or provide voice messages to a user. Information can be in the form of greetings, reminders, phone messages, and the like. Watch-like device 402 is detachable from wrist band 403 and can be attached to band 407 to be worn by a user as a necklace.

The pervasive integrated assistive technology system of this invention utilizes an application software platform to create a clinical study ecosystem for patient support, patient social contact support, support of daily activities, patient safety, symptoms management, support for caregivers, feedback/communication for clinical investigation team, and the like, in the management and execution of clinical trials to achieve superior results. The application software platform is stored in one or more servers 108, 109, 208, 209 as illustrated in FIG. 1, FIG. 2. The application software platform is accessible to users through one or more computing devices such as device 101, 102, 103, 201, 202, 203 described in this invention. Users of the application software can interact with each other via the said communication means. The software environment allows for, but is not limited to, tracking of application usage, daily tracking of participant location, monitoring medication adherence, demographics, storing and tracking health data (i.e. blood pressure, glucose, cholesterol, etc.), case report forms, audit trails, study endpoints, clinical outcomes (e.g., PROs), storing symptoms (e.g., bruising, pain), displaying symptom trends and severity, sending-receiving text messages, sending-receiving voice messages, sending-receiving videos, streaming instructional videos, scheduling clinic visits, participant education information, caregiver education information, feedback to the clinical investigation team, and the like. The application software can be used to store skills relating to self-management to comply with study objectives, protocol, and procedures. The application software may contain functions for predicting patient behaviors, functions predicting non-compliance to study protocols or procedures, functions for predicting symptom trends, functions for suggesting corrective actions, functions to perform or interventions including but not limited to correcting protocol deviation, avoiding concomitant medications. The application software may interact with an electronic health system, medical record system, other EDC applications, or other clinical study management software system.

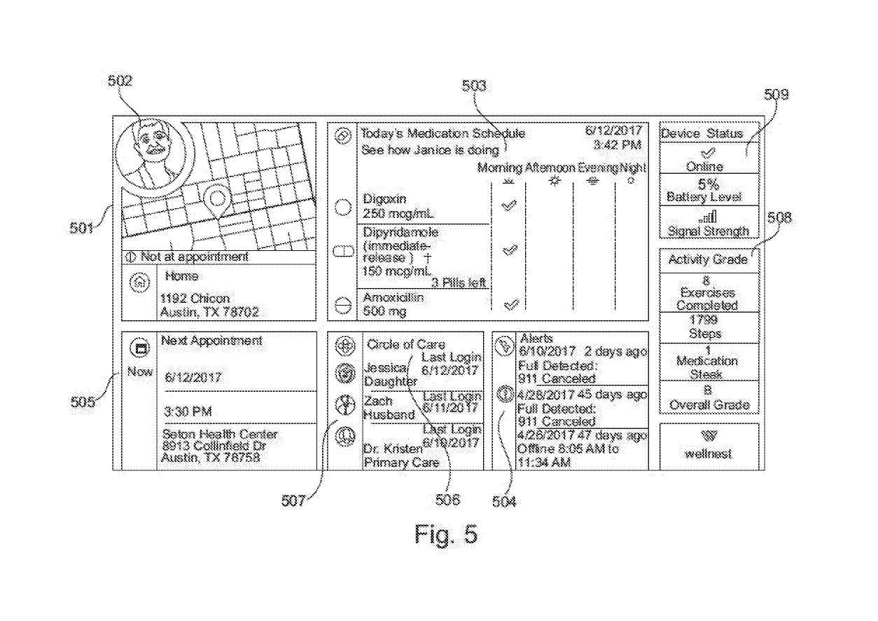

FIG. 5 is a screen-shot 501 that illustrates the type of information that users (e.g., study participant, nurse, family member) can generate using the application software platform. Screen-shot 501 provides an example of the information arranged in a specific manner and by no means limits the potential alternative or additional information that can be made available and displayed by the application software. In this example, a picture of study subject 502 is presented at the upper left corner. The application may display the current location of participant 502, providing real-time location of the subject. Knowledge of an individual's time-stamped location enables the retrieval of a host of other data types linked to geography, including elevation, temperature, sunlight/UV exposure, air quality, urban versus rural, proximity to businesses or clinical facilities, and more. Access to such integrated data may enable investigators to quantitatively measure the impact of geospatial and environmental variables on study results. A Medication Schedule 503 is available for review and contains a list of medications, dosage, and time when taken. This may be useful for caregivers and clinical investigation team members in the monitoring of patient compliance to study protocol or procedures. An Alerts 504 is also visible that documents fall detection events and 911 emergency connections for subject 502. The user can review the Next Appointment 505 information. A Circle of Care 506 has pictures of the people 507 (e.g., family members) interacting with subject 502 in this clinical study ecosystem and log-in information. There's also an Activity Grade 508 that allows users to monitor for example the physical activities of subject 502 using the step count function of the wearable device. Lastly, but not least, Device Status 509 provides information on the status of said wearable device, described for example in FIG. 3, as wearable device 301.

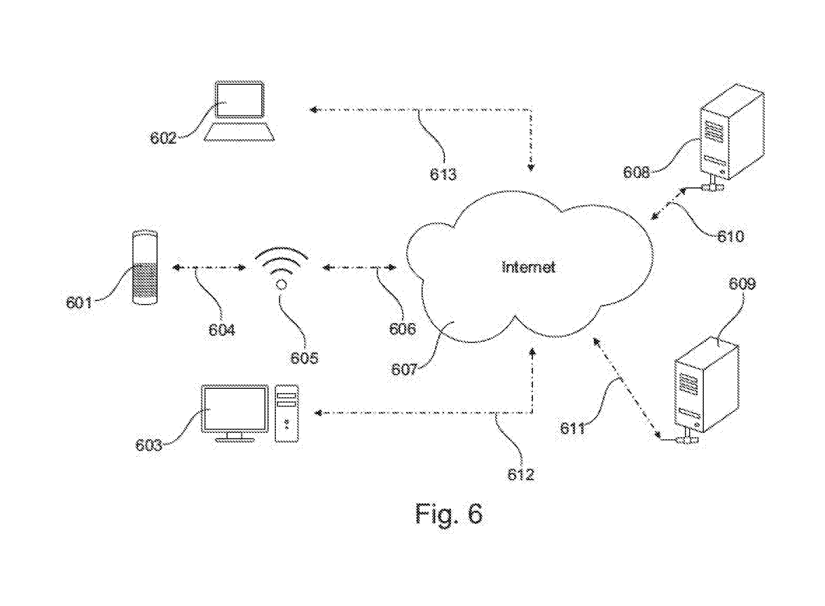

FIG. 6 illustrates the pervasive integrated assistive technology system incorporating a stand-alone voice-activated speech interface device 601 for a participant to interact with one or more remote clinical investigation team member, caregiver, or family member through a relational agent. In a similar manner as illustrated in FIG. 1, one or more user can access the system using a portable computing device 602 or stationary computing device 603. Computing device 602 may be a laptop used by a family member. Stationary computing device 603 may reside at a clinic (e.g., physician's office). Device 601 communicates with the system via communication means 604 to one or more WiFi communication network 605 which can connect device 601 via communication means 606 to the Internet 607. Device 601, 602, and 603 can access one or more remote servers 608, 609 via the Internet 607 through communication means 610 and 611 depending on the server. Device 602 and 603 can access one or more servers through communication means 612 and 613. A user may request device 601 to call a family or a clinical investigation team member (e.g., nurse Exemplary stand-alone speech interface devices with intelligent voice Al capabilities include, but not limited to: Echo, Dot, and Show; all available from Amazon (Seattle, Wash.); Siri, Duplex, Home available from Google, Inc. (Mountain View, Calif.); Cortana available from Microsoft, Inc. (Redmond, Wash.); and the like.

In a preferred embodiment, the said stand-alone device 601 enables communication with one or more remote servers, for example server 608, capable of providing cloud-based control service, to perform natural language or speech-based interaction with the user. The stand-alone speech interface device 601 listens and interacts with a user to determine a user intent based on natural language understanding of the user's speech. The speech interface device 601 is configured to capture user utterances and provide them to the control service located on server 608. The control service performs speech recognition-response and natural language understanding-processing on the utterances to determine intents expressed by the utterances. In response to an identified intent, the controlled service causes a corresponding action to be performed. An action may be performed at the control service or by instructing the speech interface device 601 to perform a function. The combination of the speech interface device 601 and control service located on remote server 608 serve as a relational agent. The relational agent provides conversational interactions, utilizing automated voice recognition-response, natural language processing, predictive algorithms, and the like, to: perform functions, interact with the user, fulfill user requests, educate user, monitor user compliance to study protocol/procedures, monitor/track user symptoms, determine user health status, user well-being, suggest corrective user actions-behaviors, and the like. The relational agent may fulfill specific requests including calling a family member, a healthcare provider, or arrange a ride (e.g., Uber, Circulation) for the user. In an emergency, for example, extreme pain, the relational agent may contact an emergency service. Ultimately the said device 601 enables the user to access and interact with the said relational agent to provide participant education, support, participant social contact support, support of daily activities, patient safety, symptoms management, support for caregivers, feedback/communication for clinical investigation team members, and the like, in the management and execution of clinical studies to achieve superior results. The information generated from the interaction of the user and the relational agent can be captured and stored in a remote server, for example remote server 609. This information may be incorporated into the application software as described in FIG. 5, making it accessible to multi-users of the clinical study ecosystem of this invention.

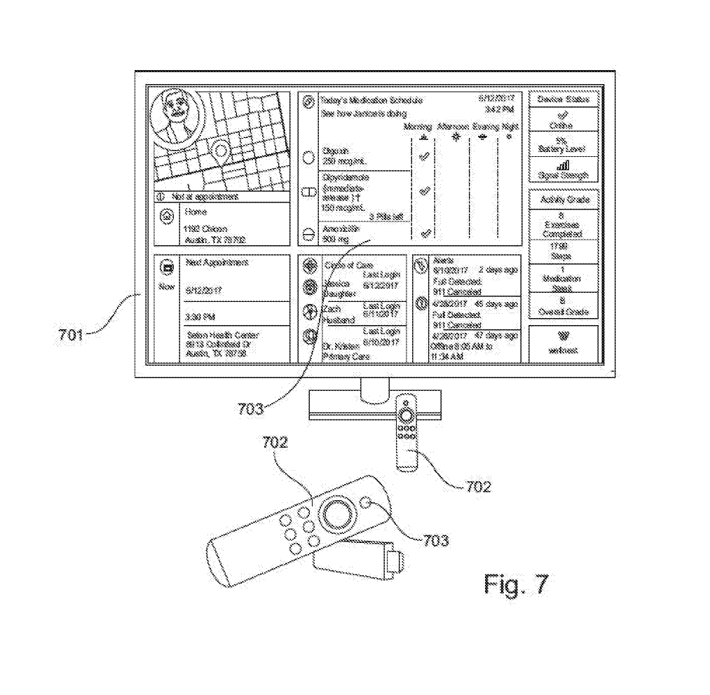

FIG. 7 illustrates the pervasive integrated assistive technology system incorporating a multimedia device 701 for a study participant to interact with one or more remote clinical investigation team member, caregiver, or family member through a relational agent. In a similar manner as illustrated in FIG. 6, one or more user can access the system using a remote-controlled device 702 containing a voice-controlled speech user interface 703. The multimedia device 701 is configured in a similar manner as device 601 of FIG. 6 as to enable a user to access application software platform depicted by screen-shot 704. The multimedia device 701 may be configured with hardware and software that enable streaming videos to be display. Exemplary products include FireTV, Fire HD8 Tablet, Echo Show; products available from Amazon.com (Seattle, Wash.), Nucleus (Nucleuslife.com), Triby (Invoxia.com), TCL Xcess, and the like. Streaming videos may include educational contents, contents about the study protocol/procedures or the like, or materials to improve participant compliance, knowledge, attitudes, and practices to improve adherence with study objectives, protocols, and procedures. Preferable materials include, but are not limited to, contents and tools to increase patient knowledge and understanding of the disease/condition relating to the study; informed consent; study objectives, protocols, or procedures; dosing regimens; symptom recognition; proper diary recording procedures; adverse event reporting; concomitant medications; nutrition; visit schedule; or the like.

In an alternative embodiment, the function of the relational agent can be accessed through a mobile app and implemented through a system illustrated in FIG. 1. Such mobile app provides access to a remote, for example remote server 108 of FIG. 1, capable of providing cloud-based control service, to perform natural language or speech-based interaction with the user. The mobile app contained in mobile device 101 monitors and captures voice commands and or utterances and transmits them through the said communication means to the control service located on server 108. The control service performs speech recognition-response and natural language understanding-processing on the utterances to determine intents expressed by the utterances. In response to an identified intent, the control service causes a corresponding action to be performed. An action may be performed at the control service or by responding to the user through the mobile app. The control service located on remote server 108 serves as a relational agent. The relational agent provides conversational interactions, utilizing automated voice recognition-response, natural language processing, predictive algorithms, and the like, to perform functions, interact with the user, fulfill user requests, educate, monitor compliance, determine health status, well-being, suggest corrective actions-behaviors, and the like. Ultimately the said device 101 enables the user to access and interact with the said relational agent for self-management during study duration. The information generated from the interaction of the user and the relational agent can be captured and stored in a remote server, for example remote server 109. This information may be incorporated into the application software as described in FIG. 5, making it accessible to multi-users of the clinical study ecosystem of this invention.

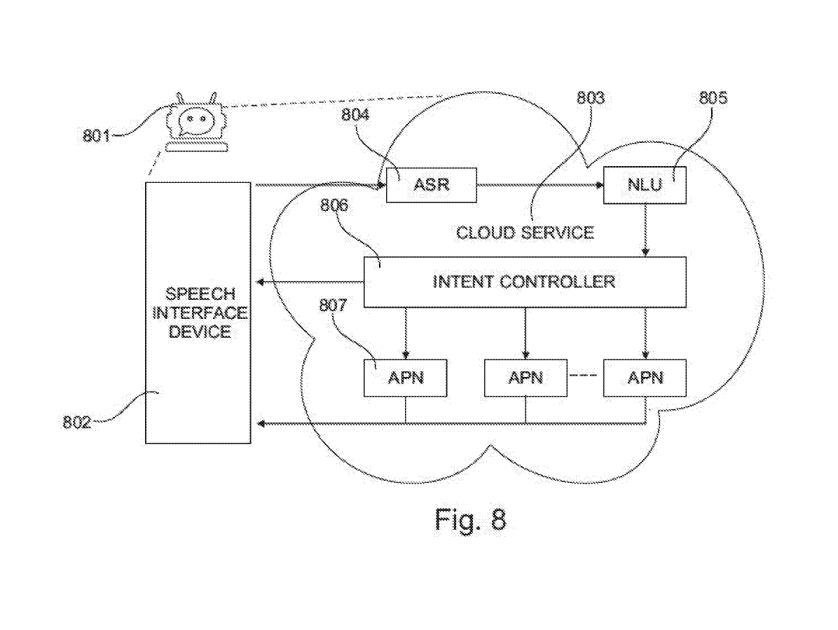

FIG. 8 illustrates a figurative relational agent 801 comprising the voice-controlled speech interface device 802 and a cloud-based control service 803. A representative cloud-based control service can be implemented through a SaaS model or the like A representative cloud-based control service can be implemented through a SaaS model or the like. Model services include, but not limited to, Amazon Web Services, Amazon Lex, Amazon Lambda, available through Amazon (Seattle, Wash.); Duplex, Home available from Google, Inc. (Mountain View, Calif.); Cortana available from Microsoft, Inc. (Redmond, Wash.); and the like. Such a service provides access to one or more remote servers containing hardware and software to operate in conjunction with said voice-controlled speech interface device, app, or the like. Without being bound to a specific configuration, said control service may provide speech services implementing an automated speech recognition (ASR) function 804, a natural language understanding (NLU) function 805, an intent router/controller 806, and one or more applications 807 providing commands back to the voice-controlled speech interface device, app, or the like. The ASR function can recognize human speech in an audio signal transmitted by the voice-controlled speech interface device received from a built-in microphone. The NLU function can determine a user intent based on user speech that is recognized by the ASR components. The speech services may also include speech generation functionality that synthesizes speech audio. The control service may also provide a dialog management component configured to coordinate speech dialogs or interactions with the user in conjunction with the speech services. Speech dialogs may be used to determine the user intents using speech prompts. One or more applications can serve as a command interpreter that determines functions or commands corresponding to intents expressed by user speech. In certain instances, commands may correspond to functions that are to be performed by the voice-controlled speech interface device and the command interpreter may in those cases provide device commands or instructions to the voice-controlled speech interface device for implementing such functions. The command interpreter can implement “built-in” capabilities that are used in conjunction with the voice-controlled speech interface device. The control service may be configured to use a library of installable applications including one or more software applications or skill applications of this invention. The control service may interact with other network-based services (e.g., Amazon Lambda) to obtain information, access additional database, application, or services on behalf of the user. A dialog management component is configured to coordinate dialogs or interactions with the user based on speech as recognized by the ASR component and or understood by the NLU component. The control service may also have a text-to-speech component responsive to the dialog management component to generate speech for playback on the voice-controlled speech interface device. These components may function based on models or rules, which may include acoustic models, specify grammar, lexicons, phrases, responses, and the like created through various training techniques. The dialog management component may utilize dialog models that specify logic for conducting dialogs with users. A dialog comprises an alternating sequence of natural language statements or utterances by the user and system generated speech or textual responses. The dialog models embody logic for creating responses based on received user statements to prompt the user for more detailed information of the intents or to obtain other information from the user. An application selection component or intent router identifies, selects, and/or invokes installed device applications and/or installed server applications in response to user intents identified by the NLU component. In response to a determined user intent, the intent router can identify one of the installed applications capable of servicing the user intent. The application can be called or invoked to satisfy the user intent or to conduct further dialog with the user to further refine the user intent. Each of the installed applications may have an intent specification that defines the serviceable intent. The control service uses the intent specifications to detect user utterances, expressions, or intents that correspond to the applications. An application intent specification may include NLU models for use by the natural language understanding component. In addition, one or installed applications may contain specified dialog models for that create and coordinate speech interactions with the user. The dialog models may be used by the dialog management component in conjunction with the dialog models to create and coordinate dialogs with the user and to determine user intent either before or during operation of the installed applications. The NLU component and the dialog management component may be configured to use the intent specifications of the applications either to conduct dialogs, to identify expressed intents of users, identify and use the intent specifications of installed applications, in conjunction with the NLU models and dialog modes, to determine when a user has expressed an intent that can be serviced by the application, and to conduct on or more dialogs with the user. As an example, in response to a user utterance, the control service may refer to the intent specifications of multiple applications, including both device applications and server applications, to identify, for example, a “Wellnest Clinical” intent. The service may then invoke the corresponding application. Upon invocation, the application may receive an indication of the determined intent and may conduct or coordinate further dialogs with the user to elicit further intent details. Upon determining sufficient details regarding the user intent, the application may perform its designed functionality in fulfillment of the intent. The voice-controlled speech interface device in combination with one or more functions 804,805,806 and applications 807 provided by the cloud service represents the relational agent 801 of the invention.

In a preferred embodiment, skills are developed for the relational agent 801 of FIG. 8 and stored as accessible applications within the cloud service 803. The skills contain information that enables the relational agent to respond to intents by performing an action in response to a natural language user input, information of utterances, spoken phrases that a user can use to invoke and intent, slots or input data required to fulfill an intent, and fulfillment mechanisms for the intent. These application skills may also reside in an alternative remote service, remote database, the Internet, or the like, and yet accessible to the cloud service 803. These skills may include but are not limited to intents for general topics, weather, news, music, pollen counts, UV conditions, participant engagement skills, skills relating to study protocol or procedures, symptom recognition and management skills, diary recording skills, skills relating to PROs, disease specific educational topics, depression, anxiety, sleep techniques, nutrition, instructions for taking medication, prescription instructions, medication adherence, persistence, coping skills, behavioral skills, daily activity, skills for collecting real-time EDC of clinical results, and the like. The skills enable the relational agent 801 to respond to intents and fulfill them through the voice-controlled speech interface device. These skills may be developed using application tools from vendors (e.g., Amazon Web Services, Alexa Skill Kits) providing cloud control services. The patient preferably interacts with relational agent 801 using skills that enable an active and collaborative effort between a study participant, clinical investigation team members, caregivers, and family members to achieve superior clinical study results.

Exemplary skills accessible to a participant may be one or more non-pharmacological interventions including CBT skills, medication adherence skills, symptom recognition skills, symptom management skills, coping skills, and the like. It is one object of this invention to provide a relational agent with skills to be able to fulfill on or more intents invoked by a subject for example; symptoms identification (e.g. pain, location, etc.) and management skills (e.g., depression, anxiety, etc.). It is a preferred object to utilize the spoken language interface as a natural means of interaction between the users and the system. Users can speak to the assistive technology similarly as they would normally speak to a human. It is understood, but not bound by theory, that verbal communication accompanied by the opportunity to engage in meaningful conversations can reinforce, improve, and motivate behavior for self-management during study participation. The relational agent may be used to engage participants in activities aimed at stimulating social functioning to leverage social support for improving study protocol/procedure compliance and persistence. These skills may create a participant-centered environment, one that is respectful of and responsive to the study subject's preferences, needs, values to encourage participant to value beneficial shared decision-making and a personal systems approach; both have the potential to improve participant compliance to achieve superior clinical study results.

The relational agent and one or more skills may be implemented in the engagement of a subject at an ambulatory setting (i.e. home, clinic, etc.). During a session, the relational agent using one or more skills may inform the subject, for example, about pain management. The patient may receive education about pain, depression, anxiety, worry, worry time, worry-free zones, insomnia, sleep strategies, procrastination, handling negative thoughts, coping strategies, stress, stress-reduction strategies, relaxation techniques, mindfulness exercises, communication skills, social skills, and the like. Skills may include instructions for recognizing adverse events and complications. The relational agent may instruct the subject about indications and models for reducing/ increasing pharmacologic interventions dosages to achieve adequate pain control. The platform of this invention preferably allows the remote monitoring by a clinical investigation team on quality (e.g. mean, standard deviation, frequency, etc.), in this example, pain symptoms and control by patients, and intervene as necessary.

It is also an object of the present invention to provide a means to record clinical outcomes using a standard set of validated questionnaires and or patient-reported outcomes (PROs) instruments. The responses-answers provided or obtained from these questionnaires and instruments enable the assessment of patient symptoms (i.e., bruising, pain), physical functioning, psychological functioning, and overall health-related QoL. One or more questionnaires and answer-responses may be self-efficacy or confidence in the ability to measure and manage the study disease-related symptoms. In a preferred embodiment on or more questionnaires in combination with answers-responses provide data for clinical study endpoints. This may be implemented using clinically validated questionnaires conducted by the relational agent. Upon a user intent, the relational agent can execute an algorithm or a pathway consisting of a series of questions that proceed in a state-machine manner, based upon yes or no responses, or specific response choices provided to the user. For example, a clinically validated structured multi-item, multidimensional, questionnaire scale may be used to assess health conditions, or symptoms, capture clinical study endpoints, and the like. The scale is preferably numerical, qualitative or quantitative, and allows for concurrent and predictive validity, with high internal consistency (i.e. high Cronbach's alpha), high sensitivity and specificity. Questions are asked by the relational agent and responses, which may be in the form of yes/no answers from patients or caregivers, are recorded and processed by one or more skills. Responses may be assigned a numerical value, for example yes=1 and no=0. One of ordinary skill in the art can appreciate the novelty and usefulness of using the relational agent of the present invention; a voice-controlled speech recognition and natural language processing combined with the utility of validated clinical questionnaire scales or PROs instruments. The questionnaire scales are constructed and implemented using skills developed through for example using the Alexa Skills Kit and or Amazon Lex. The combination of these modalities may be more conducive to eliciting information, providing feedback, actively engaging a subject during study participation, and accurately and timely capturing clinical study endpoints.

Clinically validated scales and PROs instruments may be constructed to measure, assess, or monitor, but not limited to; physical well-being, social well-being, emotional well-being, functional well-being, pain, fatigue, nausea, sleep disturbance, distress, shortness of breath, loss of memory, loss of appetite, drowsiness, dry mouth, anxiety, sadness, emesis, numbness, bruising, pain-related symptoms, or the like; rated on the basis of their presence, location, and severity. PROs instruments may also be constructed to measure, assess, or monitor medication administration, medication interactions, activity, diet, side effects, informing healthcare providers, informing clinical investigation team members, procedures, lab monitoring, and QoL. These instruments may include, but are not limited to, for example: Wisconsin Brief Pain Inventory (BPI), Pain interference (BPI-interference), Roland-Morris Disability Scale 11-item version (RMDS), Center for Epidemiological Studies Depression Scale (CES-D), Quality of Life Scale (QoLS), Pain Self-efficacy Questionnaire (PSEQ), Pain Awareness Questionnaire (PAQ), Caregiver Outcomes Assessment, Functional Assessment of Chronic Illness Therapy Fatigue, Diary: “On/off” periods, Brief Questionnaire of Smoking Urges and Minnesota Nicotine Withdrawal Scale, Diary: Nasal symptoms (runny nose, nasal itching, sneezing, and nasal congestion), Conners' Parent Rating Scale, Functional Assessment of Chronic Illness Therapy-Fatigue, Diary: Signs and symptoms of cryopyrin-associated periodic syndrome: joint pain, rash, feeling of fever/chills, eye redness/pain, and fatigue, Crohn's Disease Activity Index, Visual analogue scale-eye pain/discomfort, Diary: Urge urinary incontinence episodes and number of micturitions (frequency); International Prostate Symptom Score, Seizure frequency, Seizure severity from the Parent/Guardian Global Evaluation of the patient's condition, Pain numeric rating scale, Pain visual analogue scale, Health Assessment Questionnaire-Disability Index and Bath Ankylosing Spondylitis Functional Index, Cetirizine hydrochloride-allergy, Diary: Symptoms include sneezing, rhinorrhea, nasal pruritus, ocular pruritus, tearing, and redness of the eye, Diary: Severity and duration of hives and pruritus, Complex partial seizures-seizure frequency, Ocular itching, Mean Symptom Complex Severity and Treatment Outcome Score, Pain visual analogue scale, Toronto Western Spasmodic Torticollis (TWSTRS), Ocular itching, Activities of Daily Living and Motor subscale of the Unified Parkinson's Disease Rating Scale, Physical function, RA and PSA: Health Assessment Questionnaire, Bath Ankylosing Spondylitis Functional Index, 12-Item Multiple Sclerosis Walking Scale, Health Assessment Questionnaire, European Organization for the Research and Treatment of Cancer-Quality of Life Questionnaire Core, Short Form 36 Health Survey, Patient satisfaction (verbal rating scale), Patient global assessment of change, or the like.

It is understood that any clinically validated PROs instruments, modified or unmodified, may be implemented using the present invention. All said questionnaires, PRO instruments, scales and the like can be constructed and implemented using the Alexa Skills Kit and or Amazon Lex system, or the like. Preferable PROs instruments should possess documented content validity specific to the population condition being studied/investigated, self-administered, current recall, test-retest reliable, with proven internal consistency, with demonstrated construct validity, with proven ability to detect change, and with available responder definition. Participant responses provide real-time objective data capture of clinical study endpoints. These instruments, for example, can serve as a good quality control measure of participant compliance, measure change of internal standards, providing study data quality control. In addition, frequently missed questions may indicate potential areas for improvement in participant education, including reinforcement of clinical study guidelines as well as recommendation to contact clinical investigation team members for questions. In addition, the relational agent may assess the need for re-education or suggest areas for improvements to keep participants in compliance with study objectives, protocols, and procedures.

The said scales may be modifiable with variable number of items and may contain sub-scales with either yes/no answers, or response options, response options assigned to number values, Likert-response options, or Visual Analog Scale (VAS) responses. VAS responses may be displayed via mobile app in the form text messages employing emojis, digital images, icons, and the like.

The results from one of questionnaire, scales, and PROs instruments may be obtained and or combined to monitor and provide support for participant education, social contact, daily activities, participant safety, support for caregivers, function as a diary, serves as captured data, and feedback communication for healthcare providers, clinical investigation team members in the management and execution of clinical studies. Questionnaire, scales, and PROs instruments may be directed to either caregivers or study participants. Responses on the questionnaires are sent to the application software platform. The answers provided to the relational agent may serve as real-time data for assessment of clinical endpoints, input to one or more indices, predictive algorithms, statistical analyses, or the like, to calculate a risk stratification profile and trends. Such a profile can provide an assessment for the need of any intervention (i.e. corrective action) required by either the participants, healthcare providers, clinical investigation team members, caregivers, or family members. Trends in these symptoms can be recorded and displayed in a graphical format within the application software platform showing users (participants, caregiver, clinical investigation team member) the severity of each symptom on each day. A care team can provide personalized management recommendations for a specific patient symptom using these results. In a preferred embodiment, the clinical endpoints data are captured to support prescription claims.

In summary, the pervasive integrated assistive technology system of this invention enables a high level of interaction for participants, clinical investigation team members, caregivers, and family members to manage and execute clinical trials in an optimal manner. The system leverages a voice-activated/controlled empathetic relational agent for participant education, support, social contact support, support of daily activities, patient safety, symptoms management, support for caregivers, feedback for clinical investigation team members, and the like. For participants, the system supports the needs, but not limited to, medication adherence, symptoms management, coping, emotional support, social support, and educational information (e.g. study procedure, medication dosing regimen). For caregivers, the system supports the needs, but not limited to, information about pain medications, and their safety, advice and emotional support, and health conditions (e.g., cancer, etc.) information resources. For clinical investigation team members, the system supports the needs, but not limited to, monitoring participant compliance to instructions/protocols, patient behavior, medication adherence, routine adherence, patient health status (e.g., pain control), subject clinical data collection, and the like. The system has utility in the management and execution of clinical studies, trials, RCTs, or the like; to obtain subject data for hypotheses testing, analyses, and evaluations of safety and efficacy of therapeutic interventions (e.g., NCEs, BLAs, etc.).

### Example 1

This example is intended to serve as a demonstration of the possible voice interactions between a relational agent and a clinical study participant. The relational agent uses a control service (Amazon Lex) available from Amazon.com (Seattle, Wash.). Access to skills requires the use a device wake word (“Alexa”) as well as an invocation phrase (“Wellnest Clinical”) for skills specifically developed for a proprietary wearable device that embodies one or more components of the present invention called Wellnest Clinical Study (“WCS”). The following highlight one or more contemplated capabilities and uses of the invention:

| Feature | Sample Phrases |
|---|---|
| Onboarding | “Alexa, open WCS” (conversation will continue) |
| Demo | |
| Checking | “Alexa, ask WCS if I have any messages” “Alexa, |
| Messages | tell WCS to check my messages” |
| Appointment | “Alexa, ask WCS when my next appointment is.” |
| Schedule | “Alexa, ask WCS about my appointment schedule.” |
| Fire TV Video | “Alexa, ask WCS if there is anything new on Fire |
| Content | TV about chronic pain” |
| | “Alexa, ask WCS if there is anything new on Fire |
| | TV about opioids.” |
| | “Alexa, ask WCS if there is anything new on Fire |
| | TV about Tramadol” |
| General Help | “Alexa, tell WCS I need help to get a good night |
| | sleep.” (conversation will continue) |
| | “Alexa, tell WCS I feel anxious.” (conversation |
| | will continue) |
| Post-Study | “Alexa, ask WCS to remind me about the study |
| Initiation Visit | procedure” |
| | Alexa: “Would you like to hear instructions for the |
| | study that you are participating again?” |
| Emergency | “Alexa, tell WCS to call 911 I have extreme pain” |
| Assistance | “Alexa, ask WCS to call an ambulance” |
| Contact Family | “Alexa, tell WCS to call Hannah” |
| Medication | “Alexa, tell WCS it's morning” “Alexa, tell WCS |
| Reminders | it's midday” “Alexa, tell WCS it's evening” “Alexa, |
| | tell WCS it's nighttime” |
| Protocol | “Alexa, tell WCS I'd like to ask how/am doing with |
| Compliance/ | my medication” |
| Medication | Alexa: “I would like to ask you several questions. |
| Measure | Would you like to proceed?” |
| | Alexa: “Do you remember what you should do |
| | when realizing that you have forgotten to take your |
| | medicine?” |
| PROs measure/ | “Alexa, tell Wellnest I feel pain on my legs. Please |
| Data Collection | record the date and time.” |
| | Alexa: “I can help you record your symptoms in |
| | your diary. I would need to ask you several |
| | questions. Would you like to proceed?” |
| | Alexa: “How did you experience your pain today?” |

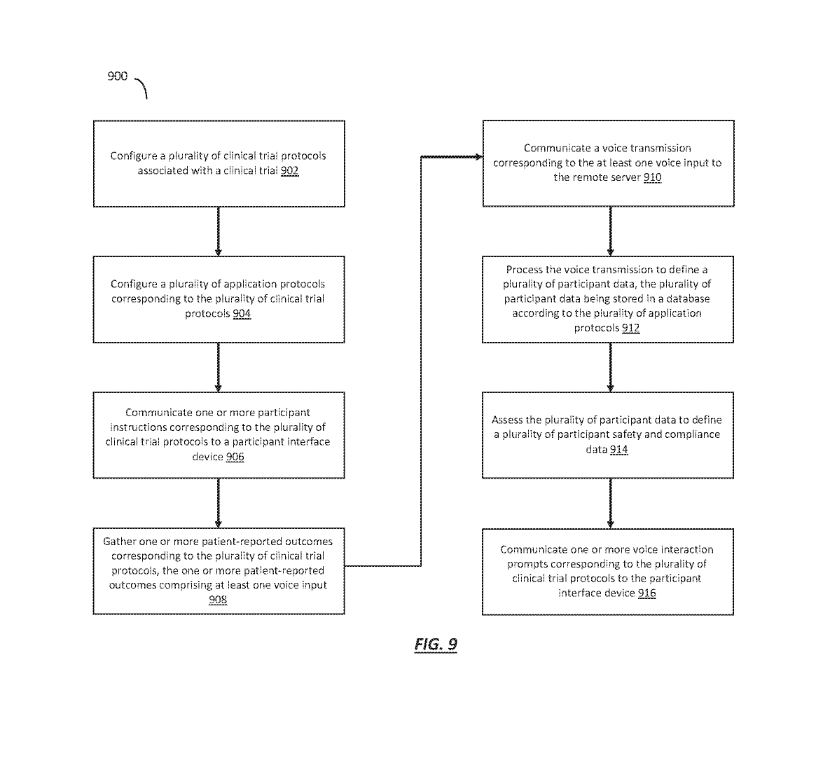

Referring now to FIG. 9, a process flow diagram of a method 900 for clinical trial management is shown. According to various embodiments of method 900, a clinical trial administrator manages a clinical trial by configuring clinical trial protocols (rules) through a web interface corresponding with a clinical trials management application executing on a remote server. The clinical trial protocols are then configured as rule, including corresponding instructions, for trial participants. Trial participants receive the clinical trial instructions to a wearable device or web/mobile interface. Throughout the duration of the clinical trial, participants can interface with the wearable device to report patient reported outcomes by a voice input, as well as receive communications from the clinical trial administrator or investigator. The patient reported outcomes are communicated from the wearable device to the application server, and the participant's voice input is then processed and stored as clinical trial data according to the clinical trial protocols. The clinical trial administrator or principal investigator can view the clinical trial data, either in a blinded or non-blinded format, throughout the trial to monitor participant safety and compliance. The clinical trial administrator (or the application software in an automated format) can deliver various voice interaction prompts to the participant throughout the trial pursuant to the clinical trial protocols. According to an embodiment of the present disclosure, a method of managing clinical trials begins with a clinical trial administrator configuring, with a clinical trial administrator interface device, a plurality of clinical trial protocols associated with the clinical trial 902. The clinical trial protocols are communicated over a network interface or communications network to a remote server executing an application software. Method 900 continues by configuring, with the remote server executing the application software, a plurality of application protocols corresponding to the plurality of clinical trial protocols 904. The remote server then communicates, via a communications network, one or more participant instructions corresponding to the plurality of clinical trial protocols to at least one participant interface device 906. During the duration of the clinical trial, the participant interface device is configured to gather one or more patient-reported outcomes corresponding to the plurality of clinical trial protocols 908. According to an embodiment, the one or more patient-reported outcomes in method step 908 correspond to at least one voice input. The participant interface device communicates, via the communications network, a voice transmission corresponding to the at least one voice input to the remote server 910. The application software executing on the remote server then processes the voice transmission to define a plurality of participant data, the plurality of participant data being stored in a database according to the plurality of application protocols 912. The application software, executing on the remote server, executes instructions to assess the plurality of participant data to define a plurality of participant safety and compliance data 914. The remote server, via the communications network, communicates one or more voice interaction prompts corresponding to the plurality of clinical trial protocols to the participant interface device.

While certain exemplary embodiments have been described and shown in the accompanying drawings, it is to be understood that such embodiments are merely illustrative of, and not restrictive on, the broad invention, and that this invention not be limited to the specific constructions and arrangements shown and described, since various other changes, combinations, omissions, modifications and substitutions, in addition to those set forth in the above paragraphs, are possible. Those skilled in the art will appreciate that various adaptations and modifications of the just described embodiments can be configured without departing from the scope and spirit of the invention. Therefore, it is to be understood that, within the scope of the appended claims, the invention may be practiced other than as specifically described herein.

What is claimed is:

1. An integrated clinical trial management system comprising:
a participant interface device operably engaged with a communications network, the participant interface device being configured to communicate a clinical trial protocol to a participant user, receive a voice input from the participant user in response to the clinical trial protocol, process a voice transmission from the voice input, and communicate the voice transmission over the communications network via at least one communications protocol;
a remote server being operably engaged with the participant interface device via the communications network to receive the voice transmission, the remote server executing a control service comprising an automated speech recognition function, a natural-language processing function, and one or more application protocols, the one or more application protocols comprising communicating a clinical trial protocol to the participant interface device, communicating instructions associated with the clinical trial protocol to the participant interface device, and storing one or more participant-reported outcomes associated with the clinical trial protocol; and,
a clinical trial administrator interface device being operably engaged with the remote server via the communications network, the clinical trial administrator interface device being operable to configure the plurality of clinical trial protocols and display the one or more participant-reported outcomes.

2. The system of claim 1 wherein the participant interface device comprises a body-worn device.

3. The system of claim 1 wherein the voice input from the participant user comprises a patient-reported outcome.

4. The system of claim 1 wherein the one or more application protocols further comprise instructions for monitoring adherence of the participant user to the plurality of clinical trial protocols.

5. The system of claim 1 wherein the participant interface device is configured to communicate one or more self-management prompts corresponding to the plurality of clinical trial protocols to the participant user.

6. The system of claim 2 wherein the one or more application protocols further comprise instructions for collecting safety data and compliance data from the participant interface device.

7. The system of claim 4 wherein the one or more application protocols further comprise instructions for communicating a participant engagement prompt to the participant interface device.

8. The system of claim 6 wherein the clinical trial administrator interface device is operably engaged with the remote server to display the safety data and compliance data in real-time.

9. An integrated clinical trial management system comprising:
a participant interface device operably engaged with a communications network, the participant interface device being configured to communicate a clinical trial protocol to a participant user, receive a voice input from the participant user in response to the clinical trial protocol, process a voice transmission from the voice input, and communicate the voice transmission over the communications network via at least one communications protocol;
a remote server being operably engaged with the participant interface device via the communications network to receive the voice transmission, the remote server executing a control service comprising an automated speech recognition function, a user management function, a natural-language processing function, and one or more application protocols, the one or more application protocols comprising instructions for communicating a clinical trial protocol to the participant interface device, communicating instructions associated with the clinical trial protocol to the participant interface device, and storing one or more participant-reported outcome associated with the clinical trial protocol;
a clinical trial administrator interface device being operably engaged with the remote server via the communications network, the clinical trial administrator interface device being operable to configure the plurality of clinical trial protocols and display the one or more participant-reported outcomes; and,
an investigator interface device being operably engaged with the remote server via the communications network, the investigator interface device being operable to configure one or more participant safety protocols.

10. The system of claim 9 wherein the participant interface device comprises a body-worn device.

11. The system of claim 9 wherein the voice input from the participant user comprises a patient-reported outcome.

12. The system of claim 9 wherein the one or more application protocols further comprise instructions for monitoring adherence of the participant user to the plurality of clinical trial protocols.

13. The system of claim 9 wherein the one or more application protocols further comprise instructions for collecting safety data and compliance data from the participant interface device.

14. The system of claim 13 wherein the one or more application protocols further comprise instructions for anonymizing and storing the safety data and compliance data.

15. The system of claim 14 wherein the investigator interface device is operably engaged with the remote server to display the anonymized safety data and compliance data in real-time.

16. A method of clinical trial management comprising:
configuring, with a clinical trial administrator interface device, a plurality of clinical trial protocols associated with a clinical trial;
configuring, with a remote server executing an application software, a plurality of application protocols corresponding to the plurality of clinical trial protocols;
communicating, with the remote server via a communications network, one or more participant instructions corresponding to the plurality of clinical trial protocols to a participant interface device;
gathering, with the participant interface device, one or more patient-reported outcomes corresponding to the plurality of clinical trial protocols, the one or more patient-reported outcomes comprising at least one voice input;
communicating, with the participant interface device via a communications network, a voice transmission corresponding to the at least one voice input to the remote server;
processing, with the application software executing on the remote server, the voice transmission to define a plurality of participant data, the plurality of participant data being stored in a database according to the plurality of application protocols;
assessing, with the application software executing on the remote server, the plurality of participant data to define a plurality of participant safety and compliance data; and,
communicating, with the remote server via the communications network, one or more voice interaction prompts corresponding to the plurality of clinical trial protocols to the participant interface device.

17. The system of claim 16 further comprising communicating, with the remote server via the communications network, the plurality of participant safety and compliance data to an investigator interface device, the plurality of participant safety and compliance data being blinded.

18. The system of claim 16 further comprising gathering, with the participant interface device, one or more participant voice interactions corresponding to the one or more voice interaction prompts.

19. The system of claim 17 further comprising gathering, with the participant interface device, one or more participant requests, the one or more participant requests comprising a participant voice input.

20. The system of claim 19 further comprising communicating, with the participant interface device via the communications network, the one or more participant requests to the investigator interface device.
